(19) United States
(12) Patent Application Publication
(10) Pub. No.: US 2013/0345330 A1
(43) Pub. Date: Dec. 26, 2013

(54) METHOD FOR PRODUCING FLEXIBLE POLYURETHANE FOAMS

(71) Applicants: Hartmut Nefzger, Pulheim (DE); Erika Bauer, Juchen (DE); Bert Klesczewski, Koln (DE); Jüergen Schlossmacher, Bergheim (DE); Sven Meyer-Ahrens, Leverkusen (DE); Manfred Schmidt, Dormagen (DE)

(72) Inventors: Hartmut Nefzger, Pulheim (DE); Erika Bauer, Juchen (DE); Bert Klesczewski, Koln (DE); Jüergen Schlossmacher, Bergheim (DE); Sven Meyer-Ahrens, Leverkusen (DE); Manfred Schmidt, Dormagen (DE)

(73) Assignee: BAYER INTELLECTUAL PROPERTY GMBH, Monheim (DE)

(21) Appl. No.: 13/988,311

(22) PCT Filed: Nov. 18, 2011
(86) PCT No.: PCT/EP2011/070481

§ 371 Date: Sep. 10, 2013

(30) Foreign Application Priority Data
Nov. 22, 2010 (EP) 10192095.7

(51) Int. Cl. C08G 18/68; C07C 67/08

(52) U.S. Cl. CPC C08G 18/68; C07C 67/08
USPC 521/157; 554/172

(57) ABSTRACT

A process for the preparation of polyricinoleic acid esters comprising the step of reaction of ricinoleic acid with an alcohol component which comprises mono- and/or polyfunctional alcohols having a molecular weight of ≧32 g/mol to ≦40 g/mol, wherein the reaction is carried out at least partly in the presence of a catalyst. The amount of catalyst, based on the total weight of the ricinoleic acid and the alcohol component, is in a range of from ≧10 ppm to ≦100 ppm. The reaction is ended when the acid number of the reaction product obtained is ≧5 mg of KOH/g to ≦100 mg of KOH/g. The invention furthermore relates to a polyurethane polymer, in particular a flexible polyurethane foam, which is obtainable using these polyricinoleic acid esters.

The present invention relates to a process for the preparation of polyricinoleic acid esters comprising the step of reaction of ricinoleic acid with an alcohol component which comprises mono- and/or polyfunctional alcohols having a molecular weight of from ≧32 g/mol to ≦400 g/mol and wherein the reaction is carried out at least partly in the presence of a catalyst. It also relates to polyurethane polymers prepared with these polyricinoleic acid esters, in particular flexible polyurethane foams.

Polyricinoleic acid esters are obtained industrially by polycondensation of ricinoleic acid. Compared with esterification of, for example, adipic acid and di-primary hydroxyl components, this reaction proceeds slowly and is therefore disadvantageous in economic terms. To compensate the substance-related reduced functionality of hydroxyl groups, a low molecular weight polyol can be added as a further component in the synthesis of polyricinoleic acid esters in order to ensure in the end the excess of hydroxyl over carboxyl groups.

For the synthesis of a polyricinoleate from ricinoleic acid and a low molecular weight polyol on an industrial scale, tank service lives of sometimes more than 80 hours are currently required in order to obtain a product having an acid number of, for example, in the region of 5 mg of KOH/g and a hydroxyl number in the region of 40 mg of KOH/g.

A preparation of polyricinoleic acid esters is described, for example, in EP 0 180 749 A1. This patent application relates to a process for the production of optionally microcellular, elastomeric shaped bodies having self-supporting properties. In this process, a reaction mixture of organic polyisocyanates and solutions of chain lengthening agents of the molecular weight range of from 62 to 400 in higher molecular weight polyhydroxy compounds of the molecular weight range of from 1,800 to 12,000 are reacted in closed moulds, with the participation of catalysts, internal mould release agents and optionally further auxiliary substances and additives.

The internal mould release agents dealt with here are condensation products which contain ester groups, are in the molecular weight range of from 900 to 4,500 and have an acid number of less than 5 and a hydroxyl number of from 12.5 to 125, obtained from 3 to 15 mol of ricinoleic acid and one mol of a mono- or polyfunctional alcohol of the molecular weight range of from 32 to 400 or in total one mol of a mixture of several such alcohols. These polyricinoleic acid esters are described as essential to the invention.

For economic reasons it would be advantageous to bring the reaction times and therefore the production costs for the synthesis of polyricinoleic acid esters to below about 30 hours. This is a typical range for other polyesters. In order to be able also to actually utilize the advantage of the shorter reaction times, it should be possible to employ such polyricinoleic acid esters as the polyol component in particular in existing flexible polyurethane foam recipes without a substantial change in the recipe. The foams produced therefrom should also be comparable with conventional foams with respect to their properties.

It has been found, surprisingly, that the abovementioned objects are achieved by a process for the preparation of polyricinoleic acid esters comprising the step of reaction of ricinoleic acid with an alcohol component which comprises mono- and/or polyfunctional alcohols having a molecular weight of from ≧32 g/mol to ≦400 g/mol and wherein the reaction is carried out at least partly in the presence of a catalyst.

The process according to the invention is distinguished in that the amount of catalyst, based on the total weight of the ricinoleic acid and the alcohol component, is in a range of from ≧10 ppm to ≦100 ppm and in that the reaction is ended when the acid number of the reaction product obtained is ≧5 mg of KOH/g to ≦50 mg of KOH/g.

Suitable mono- or polyfunctional alcohols can be, without being limited thereto, alkanols, cycloalkanols and/or polyether alcohols. Examples are n-hexanol, n-dodecanol, n-octadecanol, cyclohexanol, 1,4-dihydroxycyclohexane, 1,2-propanediol, 1,3-propanediol, 2-methylpropane-1,3-diol, 1,4-butanediol, 1,5-pentanediol, 1,6-hexanediol, 1,8-octanediol, neopentyl glycol, ethylene glycol, diethylene glycol, triethylene glycol, tetraethylene glycol, dipropylene glycol, tripropylene glycol, dibutylene glycol, tripropylene glycol, glycerol and/or trimethylolpropane.

Suitable catalysts or catalyst precursors can be Lewis or Brønsted acids, such as, for example, sulfuric acid, p-toluenesulfonic acid, tin(II) salts or titanium(IV) compounds, such as titanium tetrabutylate or titanium(IV) alcoholates.

The calculation of the catalyst content is based on the neutral compound in the case of Brønsted acids. For example, in the case of sulfuric acid the molecule H₂SO₄ is taken as the basis. If the catalyst is a Lewis acid, the catalytically active cationic species is used. For example, in the case of tin(II) salts, regardless of the particular counter-ion, only the Sn²⁺ cation or in the case of titanium(IV) compounds only the Ti⁴⁺ cation would be taken into consideration. This method of approach is advantageous, since the content of metallic species can be determined by means of atomic absorption spectroscopy (AAS) without the particular counter-ion having to be known.

The content of catalyst can also be, based on the total weight of the ricinoleic acid and the alcohol component, in a range of from ≧20 ppm to ≦80 ppm and preferably from ≧40 ppm to ≦60 ppm.

The reaction can be carried out under reduced pressure and at elevated temperature with simultaneous distilling off of the water formed during the condensation reaction. It can equally be carried out by the azeotropic method in the presence of an organic solvent, such as toluene, as an entraining agent or by the carrier gas method, that is to say by driving out the water formed using an inert gas, such as nitrogen or carbon dioxide.

According to the invention, it is envisaged that the reaction is ended when the acid number of the reaction product obtained is ≧5 mg of KOH/g to ≦50 mg of KOH/g. This value can be determined in accordance with DIN 53402 and is ascertained during the reaction, for example, by taking samples. This acid number can preferably also be in a range of from ≧5.2 mg of KOH/g to ≦20 mg of KOH/g or from ≧5.4 mg of KOH/g to ≦10 mg of KOH/g. In the simplest case, the reaction can be ended by cooling the reaction mixture, for example to a temperature of <50° C.

It has been found that the polyricinoleic acid esters (component A2) prepared according to the invention in shorter reaction times compared with the state of the art, with their comparatively high acid number and amount of catalyst, can nevertheless be advantageously used for the preparation of polyurethanes.

The molar ratio of ricinoleic acid and the alcohol component is preferably in a range of ≧3:1 to ≦10:1. Particularly preferably, this ratio is ≧4:1 to ≦8:1 and more preferably ≧5:1 to ≦7:1.

It has been found, surprisingly, that the polyricinoleic acid esters obtained by the process according to the invention can be incorporated to a particular degree into flexible foam recipes without the recipes, which were originally based on purely synthetic constituents of the polyol components, having to be changed fundamentally by the co-use of a constituent based on a natural substance (polyricinoleic acid ester), i.e. the processability and mechanical properties lie at a comparable level.

The process according to the invention preferably includes the alcohol component 1,4-dihydroxycyclohexane, 1,2-propanediol, 1,3-propanediol, 2-methylpropane-1,3-diol, 1,4-butanediol, 1,5-pentanediol, 1,6-hexanediol, neopentyl glycol, ethylene glycol, diethylene glycol, triethylene glycol, tetraethylene glycol, dipropylene glycol, cyclohexanedimethanol, glycerol and/or trimethylolpropane. 1,3-Propanediol, 1,5-pentanediol, 1,6-hexanediol, neopentyl glycol, diethylene glycol, triethylene glycol and/or trimethylolpropane are particularly preferred here. The alcohols mentioned have boiling points at which discharge together with water of reaction can be avoided and also do not tend to undergo undesirable side reactions at the conventional reaction temperatures.

The process preferably includes tin(II) salts as the catalyst. Tin(II) chloride is particularly preferred here. It has emerged that tin(II) salts do not interfere in a subsequent reaction of the polyricinoleic acid ester to give polyurethanes, or also can advantageously be employed as a catalyst in this subsequent reaction.

In the process according to the invention, the duration of the reaction is preferably ≧10 hours to ≦30 hours. Particularly preferably, the duration of the reaction is ≧12 hours to ≦25 hours and more preferably ≧15 hours to ≦20 hours.

The reaction temperature of the process is preferably ≧150° C. to ≦250° C. The temperature can also be in a range of from ≧180° C. to ≦230° C. and more preferably ≧190° C. to ≦210° C. These temperature ranges represent a good balance between the desired rate of reaction and possible undesirable side reactions, such as, for example, the elimination of water at the OH group of the ricinoleic acid.

In a preferred embodiment of the process, ricinoleic acid and the alcohol component are first reacted without the catalyst. The catalyst is then added when the water formation reaction has stopped. The reaction is then carried out further under catalysis. The fact that the reaction initially proceeds without a catalyst means that no additional external catalyst is employed. A catalysis by the constituents of the reaction mixture, polyricinoleic acid and mono- or polyfunctional alcohols themselves, is not affected by this.

The formation of water is regarded as having stopped when, according to a visual check of the reaction, no further water is distilled off or when more than 95% of the theoretical amount of water has been removed from the reaction. This can be determined, for example, by an appropriately equipped distillation receiver, a Dean-Stark apparatus or by checking the weight of the distillate formed. To determine the end of the formation of water, it is also possible, for example, to monitor the absorption properties of COOH and/or OH groups spectroscopically in the NIR range. The reaction can then be brought to completion up to previously determined absorption values.

The fact that the reaction is carried out further under catalysis after addition of the catalyst means in this connection catalysis by an added external catalyst.

According to this embodiment, a catalyst which is susceptible to hydrolysis, for example titanium(IV) alcoholates, can be employed only at a late point in time at which at least the majority of the water of reaction has already been separated off. By this means, the reaction time is not adversely influenced, since that esterification reaction proceeds under autocatalysis by the free COOH groups of the ricinoleic acid units in the initial stage and the catalyst is only introduced when the reaction mixture starts to become depleted in COOH groups.

The present invention furthermore relates to a polyricinoleic acid ester obtainable by a process according to the invention. It is distinguished by an acid number of from ≧5 mg of KOH/g to ≦50 mg of KOH/g and a catalyst content of from ≧10 ppm to ≦100 ppm. The acid number can be determined in accordance with DIN 53402. Catalysts or catalyst precursors which remain in the polyricinoleic acid ester after its preparation can be Lewis or Brønsted acids, such as, for example, sulfuric acid, p-toluenesulfonic acid, tin(II) salts or titanium(IV) compounds, such as titanium tetrabutylate or titanium(IV) alcoholates. Tin(II) salts, in particular tin(II) chloride, are preferred.

The calculation of the catalyst content is based on the neutral compound in the case of Brønsted acids. For example, in the case of sulfuric acid the molecule H₂SO₄ is taken as the basis. If the catalyst is a Lewis acid, the catalytically active cationic species is used. For example, in the case of tin(II) salts, regardless of the particular counter-ion, only the Sn²⁺ cation or in the case of Ti(IV) compounds only the Ti⁴⁺ cation would be taken into consideration. This method of approach is advantageous, since the content of metallic species can be determined by means of atomic absorption spectroscopy (AAS) without the particular counter-ion having to be known.

In one embodiment of the polyricinoleic acid ester according to the invention, this has an acid number of from ≧5.2 mg of KOH/g to ≦20 mg of KOH/g. The acid number can also be in a range of from ≧5.4 mg of KOH/g to ≦10 mg of KOH/g.

In a further embodiment of the polyricinoleic acid ester according to the invention, this has a hydroxyl number of from ≧30 mg of KOH/g to ≦80 mg of KOH/g. The hydroxyl number can be determined in accordance with DIN 53240 and can also be ≧40 mg of KOH/g to ≦60 mg of KOH/g or ≧45 mg of KOH/g to ≦50 mg of KOH/g.

In a further embodiment of the polyricinoleic acid ester according to the invention, this has a catalyst content of from ≧20 ppm to ≦80 ppm. The content can also be in a range of from ≧40 ppm to ≦60 ppm.

The present invention also provides a process for the preparation of a polyurethane polymer comprising the step of reaction of a polyisocyanate with a polyol component which comprises a polyricinoleic acid ester according to the invention.

The term “polyurethane polymer” also includes, according to the invention, prepolymers which are obtainable from the reaction of a polyisocyanate with a polyol component comprising the polyricinoleic acid ester according to the invention.

Suitable polyisocyanates (component B) are aliphatic, cycloaliphatic, araliphatic, aromatic and heterocyclic polyisocyanates such as are described e.g. by W. Siefken in Justus Liebigs Annalen der Chemie, 562, pages 75 to 136, for example those of the formula (I)

Q(NCO)ₙ (I)

in which
n=2-4, preferably 2-3,
and
Q denotes an aliphatic hydrocarbon radical having 2-18, preferably 6-10 C atoms, a cycloaliphatic hydrocarbon radical having 4-15, preferably 6-13 C atoms or an araliphatic hydrocarbon radical having 8-15, preferably 8-13 C atoms.

For example, these are those polyisocyanates such as are described in EP-A 0 007 502, pages 7-8. Preferred compounds are as a rule the polyisocyanates which are readily accessible industrially, e.g. 2,4- and 2,6-toluoylene-diisocyanate, and any desired mixtures of these isomers (“TDI”); polyphenylpolymethylene-polyisocyanates, such as are prepared by aniline-formaldehyde condensation and subsequent phosgenation (“crude MDI”) and polyisocyanates containing carbodiimide groups, urethane groups, allophanate groups, isocyanurate groups, urea groups or biuret groups (“modified polyisocyanates”), in particular those modified polyisocyanates which are derived from 2,4- and/or 2,6-toluoylene-diisocyanate or from 4,4′- and/or 2,4′-diphenylmethane-diisocyanate. Preferably, at least one compound chosen from the group consisting of 2,4- and 2,6-toluoylene-diisocyanate, 4,4′- and 2,4′- and 2,2′-diphenylmethane-diisocyanate and polyphenyl-polymethylene-polyisocyanate (“polynuclear MDI”) is employed as the polyisocyanate, and a mixture comprising 4,4′-diphenylmethane-diisocyanate and 2,4′-diphenylmethane-diisocyanate and polyphenylpolymethylene-polyisocyanate is particularly preferably employed as the polyisocyanate.

The content of polyricinoleic acid ester according to the invention in the polyol component can be, for example, ≧5% by weight to ≦60% by weight, preferably ≧10% by weight to ≦40% by weight and more preferably ≧15% by weight to ≦30% by weight.

The characteristic number (index) indicates the percentage ratio of the amount of isocyanate actually employed to the stoichiometric (NCO) amount, i.e. the amount of isocyanate groups calculated for the reaction of the OH equivalents. In the reaction mixture from which the polyurethane polymer is obtained, the characteristic number of NCO equivalents to OH equivalents can be in a range of from, for example, ≧80 to ≦120, preferably ≧85 to ≦110.

The formation of polyurethane is advantageously effected in the presence of the conventional catalysts, such as tin(II) carboxylates and/or tertiary amines

In one embodiment of this process, the polyol component furthermore comprises a conventional polyether polyol (component A1). Compounds which are called conventional polyether polyols in the context of the invention are those which are alkylene oxide addition products of starter compounds having Zerewitinoff-active hydrogen atoms, that is to say polyether polyols having a hydroxyl number according to DIN 53240 of from ≧15 mg of KOH/g to ≦80 mg of KOH/g, preferably from ≧20 mg of KOH/g to ≦60 mg of KOH/g. Starter compounds having Zerewitinoff-active hydrogen atoms which are employed for the conventional polyether polyols usually have functionalities of from 2 to 6, preferably from 3 to 6, particularly preferably of 3, and the starter compounds are preferably hydroxy-functional. Examples of hydroxy-functional starter compounds are propylene glycol, ethylene glycol, diethylene glycol, dipropylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane, triethanolamine, pentaerythritol, sorbitol, sucrose, hydroquinone, pyrocatechol, resorcinol, bisphenol F, bisphenol A, 1,3,5-trihydroxybenzene, and condensates of formaldehyde and phenol or melamine or urea containing methylol groups. Preferably, glycerol and/or trimethylolpropane is employed as the starter compound. The use of such conventional polyols based on a starter compound having a functionality of from 3 to 6 avoids the disadvantages which bifunctional polyols have, for example poor deformation values (compression set). Conventional polyether polyols based on a starter compound having a functionality of 3 are particularly preferred, since these also avoid the disadvantages originating from polyols of higher functionality, such as, for example, tetrafunctional polyols, such as their poorer elongation at break.

Suitable alkylene oxides are, for example, ethylene oxide, propylene oxide, 1,2-butylene oxide or 2,3-butylene oxide and styrene oxide. Preferably, propylene oxide and ethylene oxide are added to the reaction mixture individually, in a mixture or successively. If the alkylene oxides are metered in successively, the products prepared comprise polyether chains having block structures. Products having ethylene oxide end blocks are characterized, for example, by increased concentrations of primary end groups, which impart an advantageous isocyanate reactivity to the systems.

The invention thus also provides the production of polyurethane flexible foams having a bulk density according to DIN EN ISO 3386-1-98 in the range of from ≧10 kg/m³ to ≦150 kg/m³, preferably of from ≧20 kg/m³ to ≦70 kg/m³, and a compressive strength according to DIN EN ISO 3386-1-98 in the range of from ≧0.5 kPa to ≦20 kPa (at 40% deformation and the 4th cycle) by reaction of component A (polyol formulation) comprising
A1 50 to 95 parts by wt., preferably 50 to 80 parts by wt. (based on the sum of the parts by wt. of components A1 and A2) of conventional polyether polyol,
A2 5 to 50 parts by wt., preferably 20 to 50 parts by wt. (based on the sum of the parts by wt. of components A1 and A2) of polyricinoleic acid ester obtainable by a process comprising the step of reaction of ricinoleic acid with an alcohol component which comprises mono- and/or polyfunctional alcohols having a molecular weight of from ≧32 g/mol to ≦400 g/mol,
   wherein the reaction is carried out at least partly in the presence of a catalyst;
      characterized in that
      the amount of the catalyst, based on the total weight of the ricinoleic acid and the alcohol component, is in a range of from ≧10 ppm to ≦100 ppm, and in that
      the reaction is ended when the acid number of the reaction product obtained is ≧5 mg of KOH/g to ≦50 mg of KOH/g, preferably ≧5.2 mg of KOH/g to ≦20 mg of KOH/g, particularly preferably from ≧5.4 mg of KOH/g to ≦10 mg of KOH/g and the reaction product has a hydroxyl number of from ≧30 mg of KOH/g to ≦80 mg of KOH/g, preferably from ≧40 mg of KOH/g to ≦60 mg of KOH/g, particularly preferably from ≧45 mg of KOH/g to ≦50 mg of KOH/g.
A3 0.5 to 25 parts by wt., preferably 2 to 5 parts by wt. (based on the sum of the parts by wt. of components A1 and A2) of water and/or physical blowing agents,
A4 0.05 to 10 parts by wt., preferably 0.2 to 4 parts by wt. (based on the sum of the parts by wt. of components A1 and A2) of auxiliary substances and additives, such as
   a) catalysts,
   b) surface-active additives,
   c) pigments or flameproofing agents,
A5 0 to 10 parts by wt., preferably 0 to 5 parts by wt. (based on the sum of the parts by wt. of components A1 and A2) of compounds which contain hydrogen atoms which are reactive towards isocyanates and have a molecular weight of 62-399,
   with component B comprising polyisocyanates,
   wherein the production is carried out at a characteristic number of from 50 to 250, preferably 70 to 130, particularly preferably 75 to 115, and
   wherein all the parts by weight stated for components A1 to A5 in the present application are standardized such that the sum of the parts by weight of components A1+A2 in the composition is 100.

Components A1, A2 and B have been explained above.

### Component A3

Water and/or physical blowing agents are employed as component A3. As physical blowing agents, carbon dioxide and/or highly volatile organic substances, for example, are employed as blowing agents.

### Component A4

Auxiliary substances and additives are used as component A4, such as
a) catalysts (activators),
b) surface-active additives (surfactants), such as emulsifiers and foam stabilizers, in particular those having a low emission, such as, for example, products of the Tegostab® LF series,
c) additives such as reaction retardants (e.g. acid-reacting substances, such as hydrochloric acid or organic acid halides), cell regulators (such as, for example, paraffins or fatty alcohols or dimethylpolysiloxanes), pigments, dyestuffs, flameproofing agents (such as, for example, tricresyl phosphate), stabilizers against ageing and weathering influences, plasticizers, fungistatically and bacteriostatically acting substances, fillers (such as, for example, barium sulfate, kieselguhr, carbon black or prepared chalk) and release agents.

These auxiliary substances and additives which are optionally to be co-used are described, for example, in EP-A 0 000 389, pages 18-21. Further examples of auxiliary substances and additives which are optionally to be co-used according to the invention and details of the mode of use and action of these auxiliary substances and additives are described in Kunststoff-Handbuch, volume VII, edited by G. Oertel, Carl-Hanser-Verlag, Munich, 3rd edition, 1993, e.g. on pages 104-127.

Preferred catalysts are aliphatic tertiary amines (for example trimethylamine, tetramethylbutanediamine), cycloaliphatic tertiary amines (for example 1,4-diaza(2,2,2)bicyclooctane), aliphatic amino ethers (for example dimethylaminoethyl ether and N,N,N-trimethyl-N-hydroxyethyl-bisaminoethyl ether), cycloaliphatic amino ethers (for example N-ethylmorpholine), aliphatic amidines, cycloaliphatic amidines, urea, derivatives of urea (such as, for example, aminoalkylureas, see, for example, EP-A 0 176 013, in particular (3-dimethylaminopropylamine)-urea) and tin catalysts (such as, for example, dibutyltin oxide, dibutyltin dilaurate, tin octoate).

Particularly preferred catalysts are

α) urea, derivatives of urea and/or
β) amines and amino ethers, which in each case comprise a functional group which reacts chemically with the isocyanate. Preferably, the functional group is a hydroxyl group or a primary or secondary amino group. These particularly preferred catalysts have the advantage that these have greatly reduced migration and emission properties.

Examples of particularly preferred catalysts which may be mentioned are: (3-dimethylaminopropylamine)-urea, 2-(2-dimethylaminoethoxy)ethanol, N,N-bis(3-dimethylaminopropyl)-N-isopropanolamine, N,N,N-trimethyl-N-hydroxyethyl-bisaminoethyl ether and 3-dimethylaminopropylamine

### Component A5

Compounds which have at least two hydrogen atoms which are reactive towards isocyanates and a molecular weight of from 32 to 399 are optionally employed as component A5. These are to be understood as meaning compounds containing hydroxyl groups and/or amino groups and/or thiol groups and/or carboxyl groups, preferably compounds containing hydroxyl groups and/or amino groups, which serve as chain lengthening agents or crosslinking agents. These compounds as a rule contain 2 to 8, preferably 2 to 4 hydrogen atoms which are reactive towards isocyanates. For example, ethanolamine, diethanolamine, triethanolamine, sorbitol and/or glycerol can be employed as component A5. Further examples of compounds according to component A2 are described in EP-A 0 007 502, pages 16-17.

The present invention furthermore relates to a polyurethane polymer which is obtainable by a process according to the invention described above. The term “polyurethane polymer” also includes, according to the invention, prepolymers which are obtainable from the reaction of a polyisocyanate with a polyol component comprising the polyricinoleic acid ester according to the invention.

In a further embodiment of the polyurethane polymer, this is present as a flexible polyurethane foam. Flexible polyurethane foams in the context of the present invention are those polyurethane polymers, and in particular foams, of which the bulk density according to DIN EN ISO 3386-1-98 is in the range of from ≧10 kg/m³ to ≦150 kg/m³, preferably in the range of from ≧20 kg/m³ to ≦70 kg/m³, and of which the compressive strength according to DIN EN ISO 3386-1-98 is in the range of from ≧0.5 kPa to ≦20 kPa (at 40% deformation).

The present invention is explained further with the aid of the following examples.

### EXAMPLES

The materials and abbreviations used have the following meaning:
Ricinoleic acid: Oleo Chemie
Tin(II) chloride: Aldrich
DABCO® (triethylenediamine; 2,2,2-diazabicyclooctane): Aldrich
MDI: Mixture comprising 62 wt. % of 4,4′-diphenylmethane-diisocyanate, 8 wt. % of 2,4′-diphenylmethane-diisocyanate and 30 wt. % of polynuclear MDI having an NCO content of 32.1% by weight
PET: Polyether polyol having an OH number of approx. 28 mg of KOH/g, prepared by addition of propylene oxide and ethylene oxide in the ratio of 85 to 15 using glycerol as the starter, having approx. 85 mol % of primary OH groups
Tegostab® B 8681: Formulation of organo-modified polysiloxanes, Evonik Goldschmidt
Addocat® 105: Amine catalyst from Rheinchemie
Addocat® 108: Amine catalyst from Rheinchemie
Addocat® SO: Tin catalyst from Rheinchemie

The analyses were carried out as follows:

Dynamic viscosity: MCR 51 rheometer from Anton Paar in accordance with DIN 53019 with a CP 50-1 measuring cone (diameter 50 mm, angle 1°) at shear rates of 25, 100, 200 and 500 s⁻¹. The polyricinoleates show viscosities which are independent of the shear rate.
Hydroxyl number: with the aid of the standard DIN 53240
Acid number: with the aid of the standard DIN 53402

### A) Preparation of the Polyricinoleate

### Example A-1

### According to the Invention

343 kg of ricinoleic acid and 22 kg of hexanediol were drawn into a 500 litre stirred tank and heated to 200° C. under nitrogen and while stirring (running time approx. 2 h). In this procedure, water of reaction was initially distilled off under normal pressure. After a running time of 14 h, no further water was distilled. 65.4 g of a 28% strength solution of tin dichloride (anhydrous) in ethylene glycol were added. When the addition of the catalyst had ended, a vacuum was slowly applied to finally 30 mbar (running time to this point: 17 hours), during which the overhead temperature did not exceed 100° C. The reaction was continued at 200° C. under 30 mbar up to a total running time of 22 hours. Thereafter, the properties were determined.

Analysis of the resulting polyricinoleate A-1:
Hydroxyl number: 46.8 mg of KOH/g
Acid number: 5.42 mg of KOH/g
Viscosity: 800 mPas (25° C.)
Catalyst concentration: 33.5 ppm of tin in the end product

### Example A-2C

### Comparative Example

13,000 kg of ricinoleic acid and 650 kg of hexanediol were drawn into a 16,000 litre stirred tank with distillation columns and an attached fractionating column and heated to 200° C. under and while stirring. During the heating up phase, water of reaction was distilled off under normal pressure. When the reaction temperature was reached a vacuum was applied. The pressure was lowered to 20 mbar in the course of one hour. During this time, the overhead temperature was kept at the level of the water boiling point curve by means of regulation of the fractionating column temperature. Under a pressure of 200 mbar, 320 g of a 28% strength solution of tin dichloride (anhydrous) in ethylene glycol were added after 3.5 hours. At the same time, the fractionating column temperature was fixed at 60° C. The acid number was monitored in the course of the further reaction: The acid number was 10 mg of KOH/g after a reaction time of 24 hours in total, 5 mg of KOH/g after 48 hours, 3.5 mg of KOH/g after 72 hours and 3.0 mg of KOH/g after 84 hours. After a reaction time of 84 hours the contents of the reactor were cooled to 130° C.

Analysis of the resulting polyricinoleate A-2C:
Hydroxyl number: 37.5 mg of KOH/g
Acid number: 3.0 mg of KOH/g
Viscosity: 850 mPas (25° C.)
Catalyst concentration: 4 ppm of Sn in the end product

### Example A-3C

### Comparative Example

7,775 g of ricinoleic acid (approx. 24 mol) and 657 g (5.57 mol) of 1,6-hexanediol were initially introduced into a 10 litre four-necked flask equipped with a mechanical stirrer, 50 cm Vigreux column, thermometer, nitrogen inlet, and column head, distillation bridge and vacuum membrane pump and heated to 200° C. in the course of 60 min, while blanketing with nitrogen, water of reaction being distilled off. After 8 hours, 480 mg of tin dichloride dihydrate were added and the reaction was continued. After a reaction time of 17 hours in total, the pressure was slowly reduced to 15 mbar in the course of 5 hours. The acid number was monitored in the course of the further reaction: The acid number was 7.5 mg of KOH/g after a reaction time of 45 hours in total, 3.0 mg of KOH/g after 76 hours and 2.9 mg of KOH/g after 100 hours.

Analysis of the resulting polyricinoleate A-3C:
Hydroxyl number: 53.3 mg of KOH/g
Acid number: 2.9 mg of KOH/g
Viscosity: 325 mPas (25° C.), 100 mPas (50° C.), 45 mPas (75° C.)
Catalyst concentration: 4 ppm of Sn in the end product

TABLE 1
Overview of the polyricinoleates A-1, A-2 C and A-3 C

| Example | | A-1 | A-2C | A-3C |
|---|---|---|---|---|
| OH number | [mg of KOH/g] | 46.8 | 37.5 | 53.3 |
| Acid number | [mg of KOH/g] | 5.4 | 3 | 2.9 |
| Amount of catalyst | [ppm of zinc] | 33.5 | 4 | 4 |
| Reaction time | [hours] | 22 | 84 | 100 |
| (total) | | | | |

The technical advantage of the polyricinoleate A-1 according to the invention is clear from the greatly shortened running time compared with A-2C and A-3C.

### Examples A-4 C, A-5, A-6 and A-7

The preparation of the polyricinoleates A-4 C, A-5, A-6 and A-7 was carried out in accordance with the procedure described above for Comparative Example A-3 C, the amounts stated in Table 2 being employed. The particular reaction time and the analysis of the polyricinoleate resulting in each case are given in Table 2.

TABLE 2
Overview of the polyricinoleates A-4 C, A-5, A-6 and A-7

| | | A-4C | A-5 | A-6 | A-7 |
|---|---|---|---|---|---|
| Ricinoleic | [g] | 3,000 | 3,035 | 3,058 | 3,127 |
| acid | | | | | |
| Hexanediol | [g] | 182 | 135 | 104 | 10.6 |
| Tin(II) | [ppm] | 30 | 30 | 30 | 30 |
| dichloride | [ppm of tin] | 15.8 | 15.8 | 15.8 | 15.8 |
| dihydrate | | | | | |
| Reaction | | | | | |
| time | | | | | |
| Reaction | [hours] | 109 | 31.5 | 13 | 9.5 |
| time | | | | | |
| (total) | | | | | |
| of this, | [hours] | 85 | 22 | 5.5 | 0 |
| reaction | | | | | |
| time in | | | | | |
| vacuo | | | | | |
| Analysis | | | | | |
| Hydroxyl | [mg of KOH/g] | 38.2 | 30.8 | 31.2 | 30.0 |
| number | | | | | |
| Acid number | [mg of KOH/g] | 2.48 | 9.14 | 20 | 43.8 |
| Viscosity | [mPas, 25° C.] | 770 | 1,200 | 1,140 | 1,080 |

With the aid of the following examples (section B) it is demonstrated that the processing and the product properties of the flexible polyurethane foams produced from the polyricinoleates A-1, A-5, A-6 and A-7 according to the invention are comparable to those of flexible polyurethane foams based on A-2C, A-3C and A-4C.

### B) Production of Flexible Polyurethane Foams

The starting substances listed in the examples of the following Tables 3 and 4 are reacted with one another in the conventional method of processing for the production of flexible polyurethane slabstock foams by the one-stage process.

The characteristic number (isocyanate index) indicates the percentage ratio of the amount of isocyanate actually employed to the stoichiometric (NCO) amount, i.e. the amount of isocyanate groups calculated for the reaction of the OH equivalents:

Characteristic number=[(isocyanate amount employed):(calculated isocyanate amount)]·100 (II)

The polyurethane flexible slabstock foams obtained were subjected to a visual evaluation. The polyurethane flexible slabstock foams were classified (“foam evaluation”) with the aid of a scale of coarse-medium-fine. A classification of “coarse” here means that the foam has fewer than approx. 5 cells per cm. A classification of “medium” means that the foam has more than approx. 5 cells per cm and fewer than approx. 12 cells per cm, and a classification of “fine” means that the foam has more than approx. 12 cells per cm.

The foam quality was classified with respect to the cell structure with the aid of a scale of poor-moderate-good. A classification of “poor” here means that the foam has no uniform cell structure and/or visible defects. A classification of “moderate” means that the foam has a chiefly uniform cell structure with only few visible defects, and a classification of “good” means that the foam has a uniform cell structure without visible defects.

TABLE 3
Production and evaluation of the flexible polyurethane foams

| | | | Example | | |
|---|---|---|---|---|---|
| | | | B-1 | | B-3 |
| | | | (comp.) | B-2 | (comp.) |
| Starting | PET | [pt. by wt.] | 77.85 | 77.85 | 77.85 |
| substances | Polyol A-1 | [pt. by wt.] | | 19.46 | |
| | Polyol A-2C | [pt. by wt.] | 19.46 | | |
| | Polyol A-3C | [pt. by wt.] | | | 19.46 |
| | Water | [pt. by wt.] | 2.24 | 2.24 | 2.24 |
| | Tegostab B 8681 | [pt. by wt.] | 0.10 | 0.10 | 0.10 |
| | Addocat 105 | [pt. by wt.] | 0.16 | 0.16 | 0.16 |
| | Addocat 108 | [pt. by wt.] | 0.15 | 0.15 | 0.15 |
| | Addocat SO | [pt. by wt.] | 0.05 | 0.05 | 0.05 |
| | MDI | [WR] | 35.13 | 35.62 | 35.81 |
| | Characteristic | | 90 | 90 | 90 |
| | number | | | | |
| Processing | Start time | [s] | 10 | 10 | 12 |
| | Rising time | [s] | 120 | 130 | 125 |
| Properties | Foam | | fine | fine | fine |
| | evaluation | | | | |
| | Cell structure | | good | good | good |

Abbreviations: comp. = comparative example; pt. by wt. = parts by weight; WR = weight ratio of component A to component B at the stated characteristic number and based on 100 parts by weight of component A.

The flexible polyurethane slabstock foams into which the polyricinoleate A1 according to the invention was processed are identical to foams from the comparative example with respect to processing and properties. As can be seen, the polyol A-2C or A-3C with a synthesis duration of more than 84 hours or, respectively, 100 hours can be replaced by a polyol A-1 according to the invention with a synthesis duration of about 20 hours without changing the recipe (Table 3).

TABLE 4
Production and evaluation of the flexible polyurethane foams

| | | | Example | | | | |
|---|---|---|---|---|---|---|---|
| | | | B-4 | | | | |
| | | | (comp.) | B-5 | B-6 | B-7 | B-8 |
| Starting | PET | [pt. by wt.] | 77.85 | 77.85 | 77.85 | 77.85 | 77.74 |
| substances | Polyol A-4C | [pt. by wt.] | 19.46 | | | | |
| | Polyol A-5 | [pt. by wt.] | | 19.46 | | | |
| | Polyol A-6 | [pt. by wt.] | | | 19.46 | | |
| | Polyol A-7 | [pt. by wt.] | | | | 19.46 | 19.43 |
| | Water | [pt. by wt.] | 2.24 | 2.24 | 2.24 | 2.24 | 2.23 |
| | Tegostab B 8681 | [pt. by wt.] | 0.10 | 0.10 | 0.10 | 0.10 | 0.10 |
| | Addocat 105 | [pt. by wt.] | 0.15 | 0.15 | 0.15 | 0.15 | 0.29 |
| | Addocat 108 | [pt. by wt.] | 0.16 | 0.16 | 0.16 | 0.16 | 0.16 |
| | Addocat SO | [pt. by wt.] | 0.05 | 0.05 | 0.05 | 0.05 | 0.05 |
| | MDI | [WR] | 35.49 | 35.49 | 35.49 | 35.49 | 35.18 |
| | Characteristic | | 90 | 90 | 90 | 90 | 90 |
| | number | | | | | | |
| Processing | Start time | [s] | 10 | 10 | 12 | 12 | 10 |
| | Rising time | [s] | 120 | 140 | 175 | 195 | 135 |
| Properties | Foam | | fine | fine | fine | fine | fine |
| | evaluation | | | | | | |
| | Cell structure | | good | good | good | good | good |
| | Bulk density | [g * cm⁻³] | 59.5 | 62.7 | 63.9 | 72.6 | 67.3 |

Abbreviations: see Table 3

The flexible polyurethane slabstock foams B-5, B-6 and B-7 into which the polyricinoleates A-5, A-6 and A-7 according to the invention were processed are at a comparable level to the foams from Comparative Example B-4C with respect to the properties. As the acid number of the polyricinoleate employed increases, the rising time of the foams and the bulk density increase. As Example B-8 shows, a longer rising time and a higher bulk density can be counteracted by an increased amount of catalyst. The foams B-5, B-6, B-7 and B-8 according to the invention are identical to Comparative Example B-4C with respect to the foam evaluation and the cell structure.

1. A process for producing a flexible polyurethane foam comprising a bulk density according to DIN EN ISO 3386-1-98 in the range of from ≧10 kg/m³ to ≦150 kg/m³ and a compressive strength according to DIN EN ISO 3386-1-98 in the range of from ≧0.5 kPa to ≦20 kPa at 40% deformation and the 4th cycle, by reacting component A with component Bn
wherein component A comprises:
A1 from 50 to 95 parts by wt. based on the sum of the parts by wt. of components A1 and A2 of conventional polyether polyol,
A2 from 5 to 50 parts by wt. based on the sum of the parts by wt. of components A1 and A2 of polyricinoleic acid ester obtainable by a process comprising reacting ricinoleic acid with an alcohol component which comprises mono- and/or polyfunctional alcohols having a molecular weight of from ≧32 g/mol to ≦400 g/mol,
wherein the reaction is carried out at least partly in the presence of a catalyst; wherein
the amount of catalyst, based on the total weight of the ricinoleic acid and the alcohol component, is in a range of from ≧10 ppm to ≦100 ppm and in that the reaction is ended when the acid number of the reaction product obtained is from ≧5 mg of KOH/g to ≦50 mg of KOH/g.
A3 from 0.5 to 25 parts by wt. based on the sum of the parts by wt. of components A1 and A2 of water and/or physical blowing agents,
A4 from 0.05 to 10 parts by wt. based on the sum of the parts by wt. of components A1 and A2 of auxiliary substances and additives optionally comprising one or more
a) catalyst,
b) surface-active additive, and/or
c) pigment and/or flameproofing agent,
and wherein component B comprises one or more polyisocyanates, wherein the process is carried out at a characteristic number of from 50 to 250, and wherein all the parts by weight stated for components A1 to A5 are standardized such that the sum of the parts by weight of components A1+A2 is 100.

2. The process according to claim 1, wherein A2 comprises an acid number of from ≧5.2 mg of KOH/g to ≦20 mg of KOH/g.

3. The process according to claim 1, wherein A2 comprises a hydroxyl number of from ≧30 mg of KOH/g to ≦80 mg of KOH/g.

4. The process according to claim 1, wherein A2 is obtainable by a process having a catalyst content of from ≧20 ppm to ≦80 ppm.

5. The process according to claim 1, wherein A2 is obtainable by a process, comprising a catalyst including tin(II) salt.

6. A polyurethane polymer obtainable by a process according to claim 1.

7. A polyurethane polymer made according to claim 1, present as a flexible polyurethane foam.

8. A process for preparing polyricinoleic acid ester comprising reacting ricinoleic acid with an alcohol component which comprises mono- and/or polyfunctional alcohols having a molecular weight of from ≧32 g/mol to ≦400 g/mol,
wherein said reacting is carried out at least partly in the presence of a catalyst, wherein the amount of catalyst, based on the total weight of the ricinoleic acid and the alcohol component, is in a range of from ≧10 ppm to ≦100 ppm and further wherein
reaction is ended when the acid number of a reaction product obtained is ≧5 mg of KOH/g to ≦50 mg of KOH/g.

9. The process according to claim 8, wherein the molar ratio of ricinoleic acid and the alcohol component is in a range of from ≧3:1 to ≦10:1.

10. The process according to claim 8, wherein the alcohol component comprises 1,4-dihydroxycyclohexane, 1,2-butanediol, 1,3-propanediol, 2-methylpropane-1,3-diol, 1,4-butanediol, 1,5-pentanediol, 1,6-hexanediol, neopentyl glycol, ethylene glycol, diethylene glycol, triethylene glycol, tetraethylene glycol, dipropylene glycol, cyclohexanedimethanol, glycerol and/or trimethylolpropane.

11. The process according to claim 8, wherein said catalyst comprises tin(II) salt.

12. The process according to claim 8, wherein duration of the reaction is from ≧10 hours to ≦30 hours.

13. The process according to claim 8, wherein reaction temperature is from ≧150° C. to ≦250° C.

14. The process according to claim 8, wherein ricinoleic acid and the alcohol component are first reacted without the catalyst, the catalyst is then added when the water formation reaction has stopped and said reaction is then carried out further under catalysis.
